(19) United States
(12) Patent Application Publication
(10) Pub. No.: US 2024/0342444 A1
(43) Pub. Date: Oct. 17, 2024

(54) SOFT-TIP INTRAVASCULAR DEVICES

(71) Applicant: SCIENTIA VASCULAR, INC., West Valley City, UT (US)

(72) Inventors: John A. LIPPERT, Park City, UT (US); William Rigby ECCLES, Centerville, UT (US); Edward J. SNYDER, Park City, UT (US); Nolan LESUEUR, West Valley City, UT (US); Jorge DE FREITAS, Salt Lake City, UT (US); Brandon Rodney POPE, Salt Lake City, UT (US); Rafael MEDERO, West Valley City, UT (US)

(21) Appl. No.: 18/634,267

(22) Filed: Apr. 12, 2024

Related U.S. Application Data
(60) Provisional application No. 63/459,149, filed on Apr. 13, 2023.

(51) Int. Cl. A61M 25/09

(52) U.S. Cl. CPC A61M 25/09; A61M 2025/09075; A61M 2025/09133; A61M 2025/0915; A61M 2025/09175

(57) ABSTRACT

The present disclosure relates to guidewire devices having shapeable tips and increased flexibility. In one embodiment, a guidewire device includes a core having a proximal section and a distal section. The distal section includes a terminal section proximally adjacent to a distal end of the core, wherein the length of the terminal section is greater than the distance between a proximal end and a distal end of the terminal section. The terminal section may be curved or bent to form creases along distinct lines.

### CROSS-REFERENCE TO RELATED APPLICATIONS

This Application claims priority to and the benefit of U.S. Provisional Application No. 63/459,149, filed on Apr. 13, 2023 and titled “Soft-Tip Intravascular Devices”, the entirety of which is incorporated herein by reference.

### BACKGROUND

Guidewire devices are often used to lead or guide catheters or other interventional devices to a targeted anatomical location within a patient's body. Typically, guidewires are passed into and through a patient's vasculature in order to reach the target location, which may be at or near the patient's heart or neurovascular tissue, for example. Radiographic imaging is typically utilized to assist in navigating a guidewire to the targeted location. In many instances, a guidewire is left in place within the body during the interventional procedure where it can be used to guide multiple catheters or other interventional devices to the targeted anatomical location.

Some guidewire devices are constructed with a curved or bent tip to enable an operator to better navigate a patient's vasculature. With such guidewires, an operator can apply a torque to the proximal end of the guidewire or attached proximal handle in order to orient and point the tip in a desired direction. The operator may then direct the guidewire further within the patient's vasculature in the desired direction.

Tuning the flexibility of a guidewire device, particularly the distal sections of the guidewire device, is also a concern. In many circumstances, relatively high levels of flexibility are desirable in order to provide sufficient bendability of the guidewire to enable the guidewire to be angled through the tortuous bends and curves of a vasculature passageway to arrive at the targeted area. For example, directing a guidewire to portions of the neurovasculature requires passage of the guidewire through curved passages such as the carotid siphon and other tortuous paths.

Another concern related to guidewire devices is the ability of a given guidewire device to transmit torque from the proximal end to the distal end (i.e., the “torquability” of the guidewire device). As more of a guidewire is passed into and through a vasculature passageway, the amount of frictional surface contact between the guidewire and the vasculature increases, hindering easy movement of the guidewire through the vasculature passage. A guidewire with good torquability enables torqueing forces at the proximal end to be transmitted through the guidewire to the distal end so that the guidewire can rotate and overcome the frictional forces.

Some guidewire devices include a distally placed micro-machined hypotube positioned over the distal end of the guidewire core in order to direct applied torsional forces further distally toward the end of the device. Because torsional forces are primarily transmitted through the outer sections of a cross-section of a member, the tube is configured to provide a path for increased transmission of torque as compared to the amount of torque transmitted by a guidewire core not sheathed by a tube. Typically, such tubes are formed from a superelastic material such as nitinol so as to provide desired torque transmission characteristics in addition to providing good levels of flexibility.

While such guidewire devices have provided many benefits, several limitations remain. For example, many of the design characteristics of a guidewire having a torque-transmitting tube, although functioning to provide increased torque transmission, work against and limit the flexibility of the guidewire tip. Additionally, the curved or bent guidewire tip may become deformed as the distal end of the guidewire is pushed up against a patient's vasculature, detrimentally affecting the navigation of the guidewire device. What is needed then are configurations for increasing the flexibility and/or shapeability of the guidewire device.

### SUMMARY

The present disclosure relates to guidewire devices having shapeable tips and increased flexibility. In one embodiment, a guidewire device includes a core having a proximal section and a distal section. The distal section includes a terminal section proximally adjacent to a distal end of the core, wherein the length of the terminal section is greater than the distance between a proximal end and a distal end of the terminal section. The terminal section may be curved or bent to form creases along distinct lines.

Some embodiments further comprise a tube having a proximal section and a distal section, the tube coupled to the core such that the distal section of the core passes into and is encompassed by the tube. The distal section of the tube may be coupled to the distal section of the core at a distal attachment point disposed proximally from a distal end of the core.

In some embodiments, the tube includes fenestrations comprised of a plurality of axially extending beams and a plurality of circumferentially extending rings. The tube may include a wavy ring section wherein the rings each comprise a circumference that varies axially along a longitudinal axis of the device. The rings in the wavy ring section may include a sinusoidal pattern.

In some embodiments, the tube comprises a non-linear beam section wherein each beam includes a center line passing through the center of the beam that is not linear. For example, the beams may take the form of an “S” or a “C” shape.

In some embodiments, the tube comprises a buckling portion formed between a first and a second portion. The first portion includes a plurality of axially extending beams arranged in a first pattern with a rotational offset in a first direction, and the second portion includes a plurality of axially extending beams arranged in a second pattern with a rotational offset in a second direction opposite that of the first direction.

In some embodiments, the tube includes a thin beam section wherein a ratio of an outer diameter of the tube to a width of a beam (i.e., in the circumferential direction as opposed to the length along the longitudinal direction) is greater than or equal to about 11.3.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an exemplary embodiment of a guidewire device having a core and an outer tube and which may utilize one or more of the components described herein.

FIG. 2 illustrates an exemplary embodiment of a core of a guidewire device.

FIGS. 3A through 3D illustrate exemplary terminal sections of a distal section of the core.

FIG. 4 illustrates an exemplary embodiment of a distal section of the guidewire device, including a terminal section proximally adjacent to the distal end of the device and a distal attachment point proximally adjacent to the terminal section.

FIG. 5 illustrates an exemplary embodiment of an outer tube having a plurality of circumferentially extending rings and a plurality of axially extending beams, including a wavy portion wherein the circumferentially extending rings vary axially along the device.

FIG. 6 illustrates an exemplary embodiment of an outer tube having a plurality of circumferentially extending rings and a plurality of axially extending beams, including a non-linear beam section wherein the axially extending beams are formed in an “S” shape.

FIG. 7 illustrates an exemplary embodiment of an outer tube having a plurality of circumferentially extending rings and a plurality of axially extending beams, including a non-linear beam section wherein the axially extending beams are formed in an “C” shape.

FIG. 8 illustrates an exemplary embodiment of an outer tube having a plurality of circumferentially extending rings and a plurality of axially extending beams, including a first portion wherein a plurality of axially extending beams are arranged in a first pattern with a rotational offset in a first direction, a second portion wherein a plurality of axially extending beams arranged in a second pattern with a rotational offset in a second direction opposite that of the first direction, and a buckling portion formed between the first and second portions.

FIGS. 9A through 11B illustrate exemplary asymmetric sections of an outer tube. The asymmetric sections comprise a plurality of circumferentially extending rings and a plurality of axially extending beams, wherein the axially extending beams are disposed on a single side of the outer tube with respect to a first plane containing a longitudinal axis of the outer tube and wherein no pair of successive beams in the asymmetric section are rotationally aligned (i.e., aligned at the same circumferential position).

### DETAILED DESCRIPTION

### Example Guidewire Device Overview

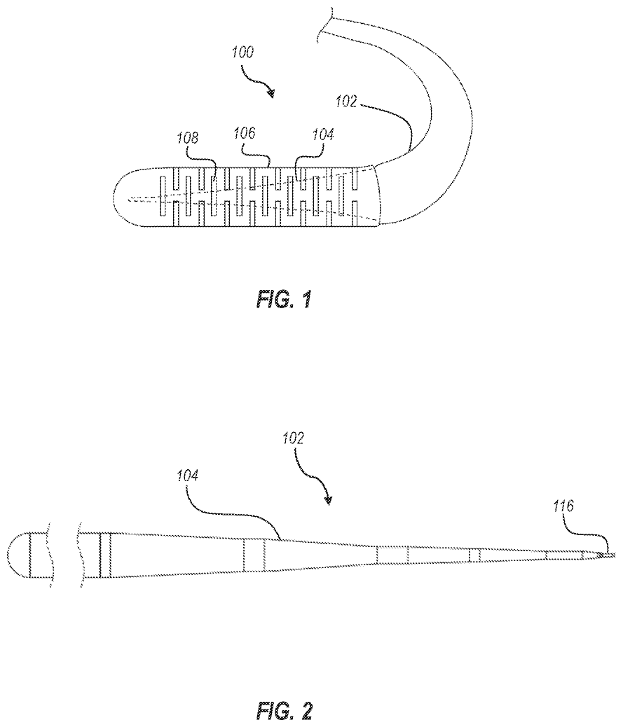

FIG. 1 schematically illustrates a guidewire device 100 suitable for utilizing one or more features of the present disclosure. The illustrated guidewire 100 includes a core 102 and an outer tube 106. The core 102 includes a distal section 104 (i.e., the distal core) that extends into the outer tube 106 as shown. The distal core 104 may be tapered, either continuously or in one or more discrete sections, so that the more distal sections have a smaller diameter and greater flexibility than more proximal sections. For example, the distal section 104 may be ground so as to progressively taper to a smaller diameter at the distal end. In some embodiments, the distal section 104 may be flattened into a ribbon-like shape with a flat, rectangular, or oblong cross section.

The core 102 and the tube 106 are typically formed from different materials. For example, the tube 106 is preferably formed from a relatively flexible and elastic material such as nitinol, whereas the core 102 may be formed from a relatively less flexible and elastic material such as stainless steel. Forming the core 102 from stainless steel may be advantageous because it allows the distal tip to hold a shape when selectively bent/shaped by an operator and because stainless steel provides sufficient modulus of elasticity to provide more responsive translational movement. While these materials are presently preferred, other suitable materials such as polymers or other metals/alloys may also be utilized.

In the illustrated device, the core 102 outer diameter and the tube 106 inner diameter have substantially similar diameters at the attachment point where the core 102 enters the tube 106. In some embodiments, the core 102 outer diameter and the tube 106 inner diameter have different diameters at the attachment point, with the difference in diameter being compensated for by a weld, solder, adhesive, or other means of structural attachment, or by positioning a portion of a centering mechanism (e.g., centering coil, braid, or tube) at the attachment point and/or through the use of a bushing structure. The tube 106 is coupled to the core 102 (e.g., using adhesive, soldering, and/or welding) in a manner that beneficially allows torsional forces to be transmitted from the core 102 to the tube 106 and thereby to be further transmitted distally by the tube 106. A medical grade adhesive or other suitable material may be used to couple the tube 106 to the core wire 102 at the distal end of the device to form an atraumatic covering.

The outer tube 106 may include a cut pattern that forms fenestrations 108 in the tube 106. The pattern of fenestrations 108 can include axially extending “beams” and circumferentially extending “rings” as shown, and/or may be arranged to provide desired flexibility characteristics to the tube 106, including the promotion of preferred bending directions, the reduction or elimination of preferred bending directions, or gradient increases in flexibility along the longitudinal axis, for example. Examples of cut patterns and other guidewire device features that may be utilized in the guidewire devices described herein are provided in detail in United States Patent Application Publication Nos. 2018/0193607 and 2018/0071496, and in Patent Cooperation Treaty Application No. PCT/US2018/034756, the entireties of each of which are incorporated herein by this reference.

The proximal section of the guidewire device 100 (the portion extending proximally from the tube 106) extends proximally to a length necessary to provide sufficient guidewire length for delivery to a targeted anatomical area. The guidewire device 100 typically has a length ranging from about 50 cm to about 350 cm depending on particular application needs. The tube 106 may have a length ranging from about 20 cm to about 65 cm, more typically about 30 cm to about 55 cm such as about 35 cm to about 45 cm.

The guidewire device 100 may have a diameter of about 0.014 inches to about 0.035 inches, though larger or smaller sizes may also be utilized depending on particular application needs, and the features of the present disclosure are not necessarily limited to certain guidewire sizes. Some embodiments may have outer diameter sizes corresponding to standard guidewire sizes such as 0.014 inches, 0.016 inches, 0.018 inches, 0.024 inches, 0.035 inches, or other such sizes common to guidewire devices. The distal section 104 of the core 102 may taper to a diameter of about 0.002 inches, or a diameter within a range of about 0.001 to 0.005 inches. In some embodiments, the distal tip may be flattened (e.g., to a rectangular cross section) to further enhance bending flexibility while minimizing reductions in cross-sectional area needed for tensile strength. In such embodiments, the cross section may have dimensions of about 0.001 inches by 0.003 inches, for example. In some embodiments, the tube 106 has a length within a range of about 3 to 100 cm.

### Example Core Features

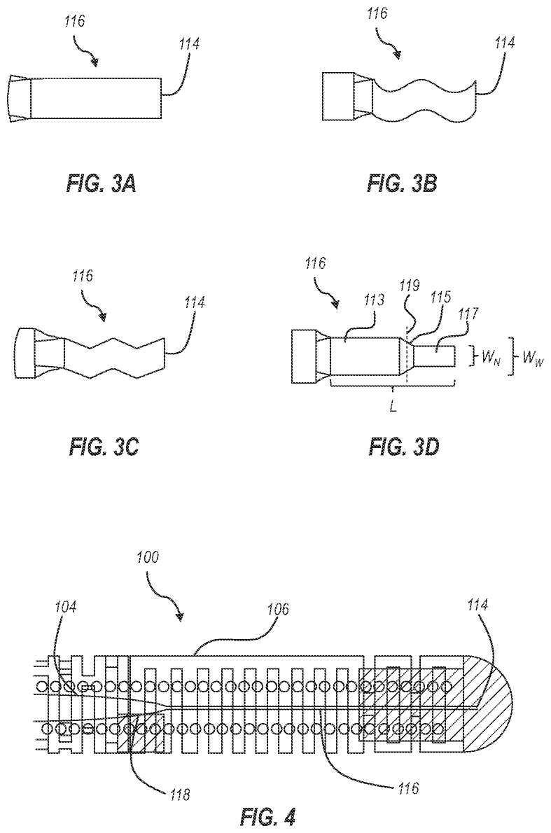

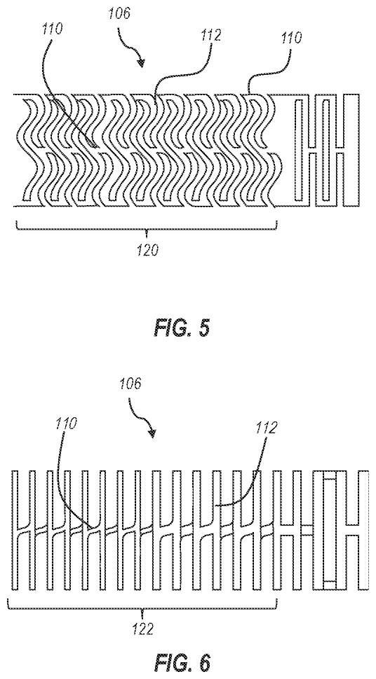

FIG. 2 illustrates an exemplary embodiment of a core 102 wherein the distal core 104 tapers in discrete sections. The distal core 104 may include a terminal section 116 disposed proximally adjacent to the distal end 114 of the core 102. The terminal section 116 may be flattened into a ribbon-like shape with a flat, rectangular, or oblong cross section. FIG. 3A schematically illustrates a terminal section 116 flattened into a ribbon-like shape and extending linearly from a proximal end of the terminal section 116 to the distal end 114 of the core 102.

In other embodiments, the terminal section 116 may be configured to reduce compressive forces delivered to the distal end of the guidewire device 100. Specifically, the core 102 may exhibit a bent and/or curved configuration such that the overall length of the material forming the terminal section 116 (i.e., the length the terminal section 116 would have if “stretched out”) is greater than the linear distance between the proximal end and the distal end of the terminal section 116.

FIGS. 3B and 3C illustrate available configurations of the terminal section 116. FIG. 3B illustrates a terminal section 116 that is curved whereas FIG. 3C illustrates a terminal section 116 that has been bent along lines running transverse to a longitudinal axis of the device 100 to form multiple creases.

These configurations promote the bending and/or flexing of the terminal section 116 when the distal end of the guidewire device 100 is pushed up against obstacles, such as endovascular tissue, as a result of pushing the guidewire device along the patient's vasculature. A bent or curved terminal section 116 enables the distal section of the core to store compressive energy elastically in the terminal section 116 rather than deform the tip of the guidewire device 100. Such configurations thereby aid in maintaining the preferred shape of the tip of the guidewire device 100.

FIG. 3D illustrates another configuration of the terminal section 116. In this embodiment, the terminal section 116 comprises, sequentially from a proximal to distal direction, a wide portion 113, a tapered portion 115, and a narrow portion 117. The wide portion 113 has a width “W_{W}” greater than the width “W_{N}” of the narrow portion 117. The flattened terminal section 116 is shapeable and in some embodiments functions as the primary shapeable portion of the core. The “double flat” configuration shown in FIG. 3D beneficially provides structure for shaping (particularly from the wide portion 113), while also providing an intentional stress point due to the transition between the wide portion 113 and the narrow portion 117 conductive to buckling.

The terminal section 116 of FIG. 3D may have a circular cross section, or a cross section having a different shape, but preferably is formed into a flat ribbon having a rectangular or oblong cross section. The terminal section 116 has a length “L”, comprising the distance from the proximal end of the wide portion 113 to the distal end of the narrow portion 117. The length “L” of the terminal section 116 may be approximately 0.5 cm to approximately 3 cm, or approximately 0.75 cm to approximately 2 cm, or may extend approximately 1 cm, or may extend within a range having as endpoints any two of the foregoing values.

The tapered portion 115 is situated within the terminal section 116 so as to be off-center from a center point lying along the proximal-to-distal axis of the device within length “L” of the terminal section 116. That is, a plane 119 oriented perpendicular to the proximal-to-distal axis of the core passing through the center of the tapered portion 115 does not intersect a center point on the proximal-to-distal axis of the core lying halfway between the proximal end of the wide portion 113 and the distal end of the narrow portion 117.

Placement of the tapered portion 115 off-center of the terminal section 116 increases the likelihood that the terminal section 116 will buckle at the tapered portion 115 when subjected to a given force. The off-center configuration reduces the amount of force required to induce buckling in the terminal section. That is, axial forces required to cause buckling at an off-center segment of an elongated member are smaller than the axial forces required to cause buckling at the center of a similar elongated member, thus reducing the likelihood of transmitting forces within the patient vasculature sufficient to permanently deform the shaped guidewire tip or to damage intravascular tissue.

The wide portion 113 and the narrow portion 117 have different lengths to ensure that the tapered portion 115 is situated off-center within the terminal section 116. The narrow portion 117 may have a longer or shorter length than the length of the wide portion 113. Preferably, the wide portion 113 has a greater length than the narrow portion 117. A configuration with a relatively longer wide portion 113 can provide effective shapeability functions while also allowing sufficient length of the narrow portion 117 to decrease forces required for buckling.

The narrow portion 117 may preferably extend from approximately 10% to less than 50% of the length “L” of the terminal section 116, preferably having a length of at least 5 mm. Conversely, the wide portion 113 may preferably extend from greater than 50% to less than 90% of the length “L” of the terminal section 116. The terminal section 116 having the configuration described above beneficially has a good propensity to maintain a shaped guidewire tip.

### Example Tube Features

FIG. 4 illustrates a distal end of an exemplary guidewire device 100. The distal section of the outer tube 106 may be bonded to the core 102 at a distal attachment point 118 disposed proximally of the terminal section 116. The distal end 114 of the core 102 may be left unbonded. This bonding configuration enables the terminal section 116 to bend and flex within the annular space of the outer tube 106 when forces are applied to the distal end 114 of the core 102.

When the distal end of the guidewire device 100 comes into contact with endovascular tissue, compressive energy induced in the guidewire device (as a result of technical manipulation of the device 100) may similarly be stored in the terminal section 116 as it bends and flexes. This configuration enables the terminal section 116 to store compressive energy elastically rather than in deformation of the tip of the guidewire device 100, thus preventing deformation of a guidewire tip shaped to effectively navigate the contours of a patient's vasculature. A terminal section capable of independently bending and flexing also helps to prevent the transmission of relatively large forces from the guidewire to the patient's vasculature, thus reducing the likelihood of harm and injury to tissue that would result from the application of the compressive forces.

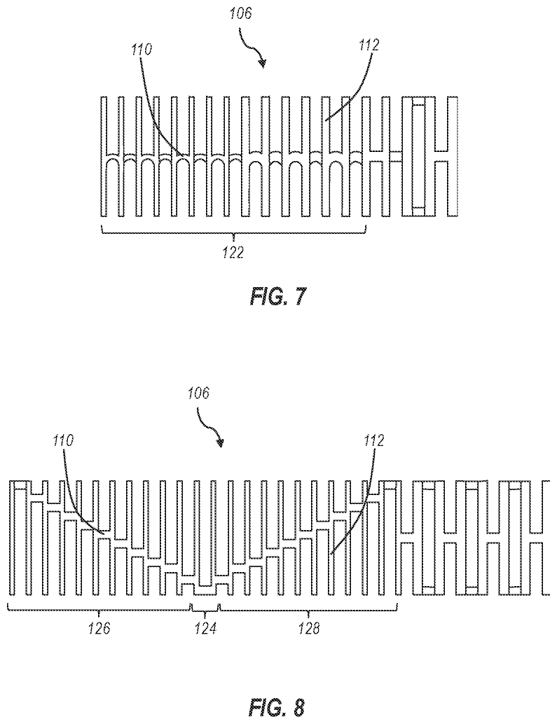

The following embodiments refer to outer tubes 106 configured to increase the flexibility and/or alternatively induce or eliminate preferred bending directions in the guidewire device 100. FIG. 5 illustrates an exemplary embodiment of an outer tube 106, including a wavy ring section 120. The wavy ring section 120 includes circumferentially extending rings 112 whose circumference varies axially along the device 100.

Each ring 112 may vary axially at the same rate as other rings 112 disposed proximally and/or distally adjacent, such that the shape of each ring 112 conforms to the shape of other adjacent rings 112. Additionally, the wavy ring section 120 may comprise a one beam, two beam, or three beam cut pattern. Successive beams 110 may be rotationally offset by 90 degrees, especially in the case of one or two beam cut pattern embodiments. These configurations enable the outer tube 106, while bending, to eliminate to a greater degree space between the rings 112 on the side of the tube 106 in the bending direction. In this manner the rings 112 may be packed closer together on the same side of the outer tube 106 as the bending direction, enabling the outer tube 106 to bend at a faster rate and increasing the flexibility of the device 100.

The wavy ring section 120 may be formed to include a sinusoidal shape along its circumference, forming a wave pattern. A line traveling along the circumference of a ring 112 may include one or more inflection points (i.e., locations along a line in which a change in direction of curvature occurs).

The outer tube 106 may also comprise a non-linear beam section 122 to further increase flexibility of the device 100. The axially extending beams 110 of the non-linear beam section are configured such that a line passing through the center of the beam 110 (i.e., a centerline) is not linear. For a beam 110 traversing a pair of rings 112 spaced at a particular distance, a non-linear beam 110 will have a greater effective length than a linear beam 110. Thus, a non-linear beam 110 will exhibit increased flexibility over a linear beam 110 because it has a greater length over which to flex and bend.

For example, the beams 110 may be formed in an “S” shape such that the centerline of the beam 110 includes an inflection point, as shown in FIG. 6. Additionally, the beams 110 may be formed in a “C” shape such that a centerline of the beam 100 includes a turning point (i.e., a point along a line indicating a local maximum or minimum in which the line changes direction), as shown in FIG. 7. The non-linear beam section 122 may also be arranged in one beam, two beam, or three beam cut configurations.

“S” and “C” shaped beams 110 may also aid in inducing or eliminating preferred bending directions. The circumferentially extending rings 112 may have a greater tendency to tilt in directions exhibiting less material support from the beams 110. Compared to linear beams 110, “S” and “C” shaped beams 110 distribute material from one side of the beam 110 to another side at both ends of the beam 110. This results in rings 112 having a greater tendency to tilt in directions from which material has been comparatively removed and aids in inducing or eliminating a preferred bending direction.

Other embodiments of the outer tube 106 may include geometries for inducing bending or buckling in a preferred orientation. FIG. 8 illustrates an exemplary embodiment of an outer tube 106 including a buckling portion 124. The buckling portion 124 is bounded proximally by a first portion 126 and bounded distally by a second portion 128. The first portion 126 includes a plurality of axially extending beams 110 arranged in a first pattern with a rotational offset in a first direction. The second portion 128 includes a plurality of axially extending beams 110 arranged in a second pattern with a rotational offset in a second direction.

The beams 110 of the buckling portion 124 may be disposed on one side of the outer tube 106 with respect to the longitudinal axis of the outer tube 106. The buckling portion 124 may include one beam 110 or multiple successive beams 110, such as two, three, or more than three successive beams 110. The configuration of beams 110 within the first 126, second 128, and buckling 124 portions assist in defining the bending direction of the tube 106 in the event of buckling, with the bending direction on a side opposite the beams 110 of the buckling portion 124.

The first 126 and second 128 portions may comprise linear patterns wherein successive beams 110 are rotationally offset by a consistent amount, as shown in FIG. 8. Alternatively, the first 126 and second 128 portions may comprise patterns wherein successive beams 110 are rotationally offset by a changing amount, such as a pattern wherein the rotational offset of successive beams 110 increases distally along the outer tube 106 or wherein the rotational offset of successive beams 110 decreases distally along the outer tube 106. Additionally, the amount of rotational offset between successive beams 110 in a linear pattern may be different in that of the first portion 126 compared to the second portion 128. Similarly, the rate of rotational offset of a non-linear pattern may be different in the first portion 126 compared to the second portion 128.

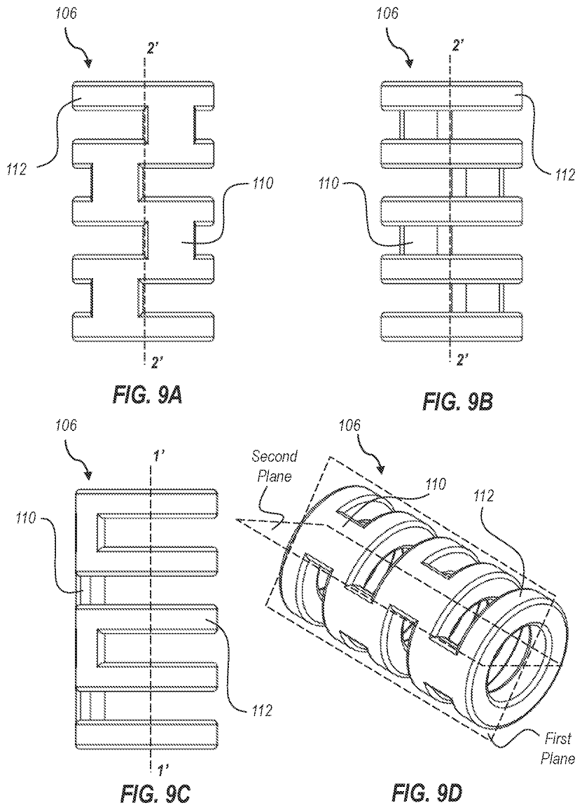

Other embodiments of an outer tube 106 may include an asymmetric section. Exemplary embodiments of asymmetric sections are illustrated in FIGS. 9A through 11B. Within the asymmetric section, the axially extending beams 110 are disposed on one side of the outer tube 106 with respect to a first plane (see FIG. 9D) that contains the longitudinal axis and extends through the center of the outer tube 106. The arrangement of the beams 110 in the asymmetric section may create a preferred bending direction toward the side of the outer tube 106, relative to the first plane, that includes the beams 110.

The beams 110 of the asymmetric section are arranged such that no pair of successive beams 110 are rotationally aligned. Specifically, a centerline running through the center of each beam 110 is rotationally offset between a proximal-distal pair of beams 110.

The beams 110 of the asymmetric section may be arranged in a one beam pattern, the beams 110 arranged so that successive beams 110 are disposed alternately on either side of a second plane, the second plane disposed perpendicular to the first plane and also containing the longitudinal axis (see FIG. 9D). In such an arrangement, each pair of successive beams 110 may be offset to opposite sides of the second plane in an alternating fashion.

FIGS. 9A through 9D illustrate asymmetric sections of an outer tube 106, the beams 110 disposed adjacent the second plane, with successive beams 110 alternatingly positioned to either side of the second plane. Lines 1′-1′ and 2′-2′ represent the side profiles of the first and second planes. FIG. 9A illustrates a front view of the outer tube 106 while FIG. 9B illustrates a back view. FIG. 9C illustrates a side view of the asymmetric section with all beams 110 disposed to one side of the outer tube 106 with respect to the first plane. FIG. 9D illustrates a perspective view of the asymmetric sections wherein successive beams 110 are alternatingly disposed adjacent to the second plane.

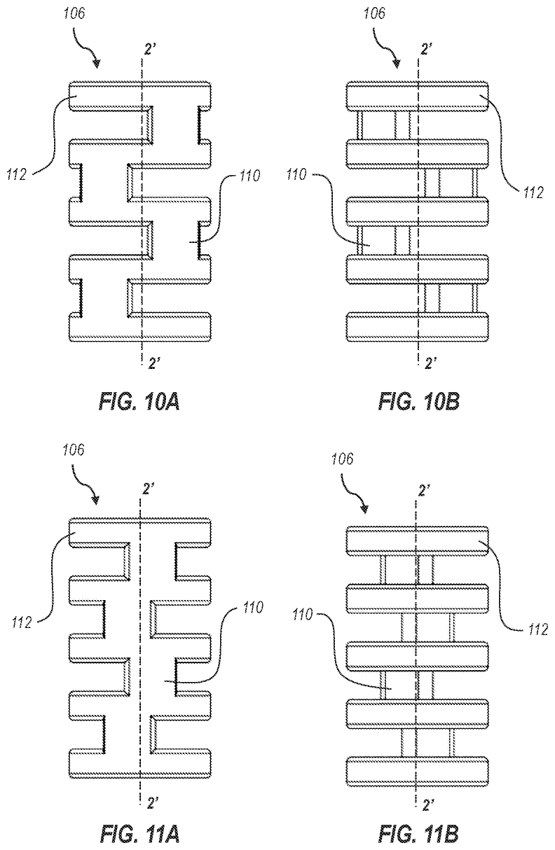

The beams 110 may be spaced a distance from the second plane, in an alternating fashion. FIGS. 10A and 10B illustrate such an arrangement, the beams 110 spaced farther apart from the second plane compared to the configuration of FIGS. 9A through 9D. FIGS. 11A and 11B illustrate an additional arrangement in which the beams 110 of the asymmetric section may intersect the second plane. The beams 110 may be arranged such that each beam 110 intersects the second plane, but with the centerline of each successive beam 110 still being rotationally offset from the second plane in an alternating fashion.

The flexibility of the distal section of the outer tube 106 may be optimized by decreasing the width of the beam 110 relative to the outer diameter of the tube 106. Specifically, the distal section of the outer tube 106 may attain increased flexibility when the ratio between the outer diameter of the tube 106 and the width of the axially extending beams 110 in the distal section of the tube 106 is greater than or equal to about 11.3.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Note that while the term “core” can be taken to mean a solid wire structure, the term as used herein is inclusive of inner members that do not necessarily require a solid structure.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. An intravascular device, comprising:
a core having a terminal section adjacent to a distal end of the core, wherein the terminal section includes
a wide portion,
a tapered portion connected to a distal end of the wide portion and having a width that tapers in a proximal to distal direction, and
a narrow portion connected to a distal end of the tapered portion,
wherein the wide portion has a width greater than the width of the narrow portion, and
wherein the narrow portion has a shorter length than the wide portion.

2. The interventional device of claim 1, wherein the narrow portion has a length of 10% to less than 50% of the length of the terminal section.

3. The interventional device of claim 1, wherein the terminal section has a length of approximately 0.5 cm to approximately 3 cm.

4. The intravascular device of claim 1, further comprising:
a tube having a proximal section and a distal section, the tube coupled to the core such that a distal section of the core passes into and is encompassed by the tube,
wherein the distal section of the tube is coupled to the distal section of the core at an attachment point disposed proximally from a distal end of the core, and
wherein the distal end of the core is not coupled to the tube.

5. The intravascular device of claim 4, wherein the attachment point is disposed approximately 1 cm to approximately 5 cm from the distal end of the core.

6. The intravascular device of claim 4, wherein the tube comprises:
a wall and an interior lumen,
a plurality of fenestrations extending through the wall and exposing the lumen, the plurality of fenestrations defining a plurality of axially extending beams and a plurality of circumferentially extending rings, and
a wavy ring section, wherein the rings of the wavy ring section each comprise a circumference that varies axially along a longitudinal axis of the device.

7. The intravascular device of claim 6, wherein at least one of the rings of the wavy ring section comprises a circumference that includes a sinusoidal pattern along a longitudinal axis of the device.

8. The intravascular device of claim 6, wherein a line tracing a circumference of at least one ring within the wavy ring section includes multiple inflection points.

9. The intravascular device of claim 8, wherein the tube comprises a non-linear beam section, wherein each beam of the non-linear beam section extends such that a center line passing though the center of the beam is not linear.

10. The intravascular device of claim 9, wherein the center line of at least one beam takes the form of an “S” shape, such that the center line includes an inflection point, or a “C” shape, such that the center line includes a turning point.

11. The intravascular device of claim 6, wherein the tube further comprises:
a first portion having a plurality of axially extending beams arranged in a first pattern with a rotational offset in a first direction,
a second portion having a plurality of axially extending beams arranged in a second pattern with a rotational offset in a second direction opposite that of the first direction, and
a buckling portion formed between the first and second portions.

12. The intravascular device of claim 6, wherein the tube further comprises an asymmetric section in which the beams are disposed on a single side of the tube with respect to a first plane containing a longitudinal axis of the tube, and wherein no pair of successive beams in the asymmetric section are circumferentially aligned.

13. The device of claim 12, wherein the beams of the asymmetric section are arranged in a one-beam pattern.

14. The device of claim 13, wherein the beams of the asymmetric section are arranged to either side of a second plane, the second plane containing the longitudinal axis and being perpendicular to the first plane, the successive beams disposed in an alternating fashion on alternating sides of the second plane.

15. The intravascular device of claim 6, wherein the tube comprises a thin beam section in which a ratio of an outer diameter of the tube to a width of corresponding beams is greater than or equal to 11.3.

16. An intravascular device, comprising:
a core having a proximal section and a distal section, the distal section including a terminal section proximally adjacent to a distal end of the core,
wherein the terminal section includes one or more creases and/or curves such that an overall length of material forming the terminal section is greater than a linear distance between a proximal end and a distal end of the terminal section.

17. The intravascular device of claim 16, wherein the terminal section is bent along one or more lines running transverse to a longitudinal axis of the core to form the one or more creases in the terminal section.

18. An interventional device, comprising:
an elongated member having a wall and an interior lumen, the elongated member including a plurality of fenestrations extending through the wall and exposing the lumen, the plurality of fenestrations defining a plurality of axially extending beams and a plurality of circumferentially extending rings,
wherein the elongated member comprises a wavy ring section,
wherein the rings of the wavy ring section each comprise a circumference that varies axially along a longitudinal axis of the device.

19. The device of claim 18, wherein a line tracing a circumference of at least one ring within the wavy ring section includes multiple inflection points.

20. The interventional device of claim 18, wherein the elongated member comprises a non-linear beam section, wherein each beam of the non-linear beam section extends such that a center line passing through the center of the beam is not linear.
